(19) United States
(12) Plant Patent Application Publication
(10) Pub. No.: US 2026/0182481 P1
(43) Pub. Date: Jun. 25, 2026

(54) Strawberry Plant Named 'CBC008'

(50) ;

(71) Applicant: Kyle M. VandenLangenberg, Tracy, CA (US)

(72) Inventor: Kyle M. VandenLangenberg, Tracy, CA (US)

(73) Assignee: California Berry Cultivars, LLC, French Camp, CA (US)

(21) Appl. No.: 19/427,767

(22) Filed: Dec. 19, 2025

Related U.S. Application Data
(60) Provisional application No. 63/738,089, filed on Dec. 23, 2024.

(51) Int. Cl. A01H 6/74; A01H 5/08

(52) U.S. Cl. CPC A01H 6/7409; A01H 5/08
USPC PLT/208

(57) ABSTRACT

A new and distinct cultivar of strawberry plant (Fragaria x ananassa) named ‘CBC008’ is presented here. This new day-neutral strawberry cultivar is characterized by moderate vigor plants which produce high yields of large, firm, flavorful fruit throughout the growing season.

Latin name of the genus and species of the plant claimed:

Botanical classification: Fragaria x ananassa.

Variety demonation:

The new strawberry variety denomination is ‘CBC008’.

### BACKGROUND OF THE INVENTION

This invention relates to a new and distinctive day-neutral type cultivar designated as ‘CBC008’. The new cultivar was the result of a controlled cross performed in 2015 in Huelva, Spain between the cultivar ‘Sabrina’ (U.S. Plant Pat. No. 22,506) and the cultivar ‘Driscoll Camarillo’ (U.S. Plant Pat. No. 14,771). ‘CBC008’ was first fruited at an experimental orchard near French Camp, California in 2016, where it was selected, originally designated ‘115.079-101’, and propagated asexually by runners. Following selection and during testing, the plant of this selection was later designated ‘CBC008’. Asexual propagules from this original source have been tested in Watsonville, California, in Oxnard, California, and to a limited extent in grower fields starting in 2020. The cultivar is stable and reproduces true to type in successive generations of asexual reproduction.

### COMPARISON WITH PARENTS

The parent ‘Sabrina’ is a short-day cultivar that was selected for its overall high appearance score and large, firm fruit. Plants of ‘Sabrina’ are larger than ‘CBC008’. The parent ‘Dricoll Camarillo’ is a day-neutral cultivar that was selected for its high early season yield. Plants of ‘Driscoll Camarillo’ are similar in size to ‘CBC008’.

### SUMMARY OF THE INVENTION

‘CBC008’ is a day-neutral cultivar that produces fruit over an extended period when treated appropriately in arid, sub-tropical climates. When treated with appropriate planting regimes, ‘CBC008’ has larger fruit and produces individual plant yields greater than that of either parent. The following traits have been repeatedly observed over multiple testing years and locations and are determined to be unique characteristics of ‘CBC008’. These characteristics in combination distinguish ‘CBC008’ as a new and distinct Strawberry plant:
1. Compact to medium sized plant.
2. Day-neutral flowering habit.
3. Medium glossy red fruit.
4. Fruit is firm throughout the growing season.
5. Fruit postharvest longevity is good.
6. Excellent disease resistance.

The cultivar ‘CBC008’, when planted under appropriate conditions, is a continuously flowering day-neutral type with exceptionally high total yields. Fruit quality is good with excellent flavor. In addition to its yield performance, ‘CBC008’ is resistant to Macrophomina phaseolina, the causal agent of charcoal rot, Fusarium oxysporum f. sp fragariae, the causal agent of Fusarium wilt, and Verticillium dahlia, the causal agent of verticillium wilt.

### BRIEF DESCRIPTION OF THE FIGS.

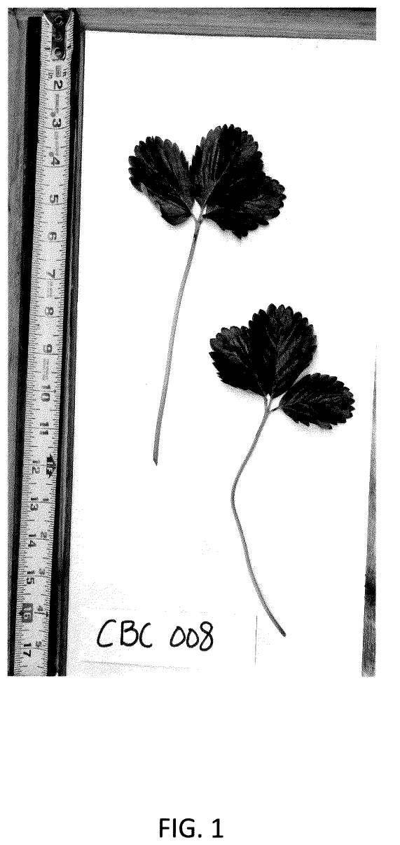

The accompanying color photographs, identified as FIGS. 1 through 5, show the appearance of typical specimens of the new strawberry cultivar designated ‘CBC008’. These Figures depict the colors, as nearly true as can be achieved with conventional photography.

FIG. 1 shows a typical leaf and petiole structure.

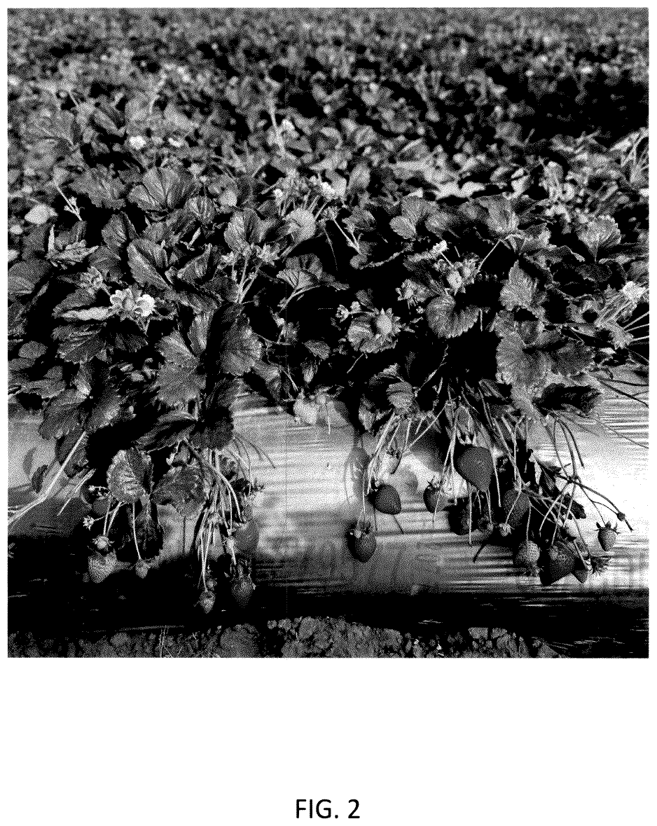

FIG. 2 shows a typical plant and fruit early season.

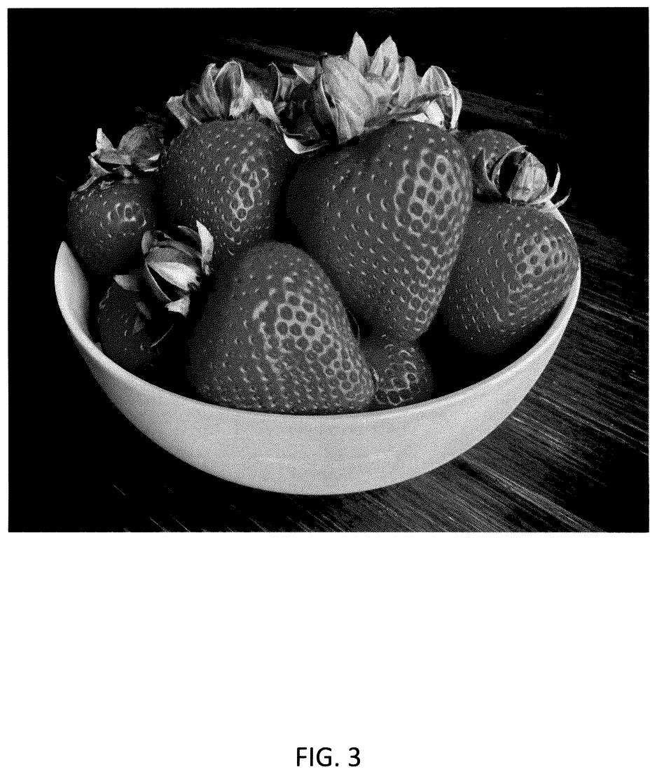

FIG. 3 shows typical fruit in a bowl.

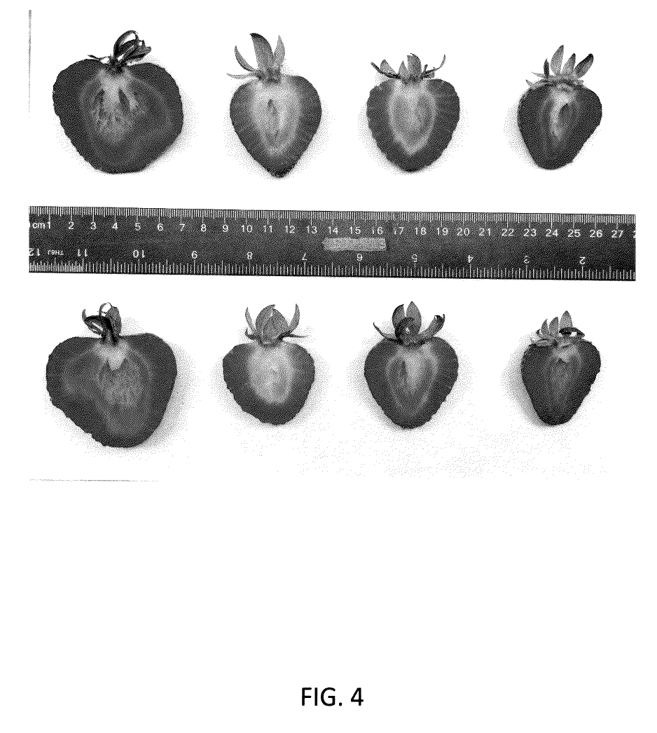

FIG. 4 shows a cross-section of representative fruit mid-season.

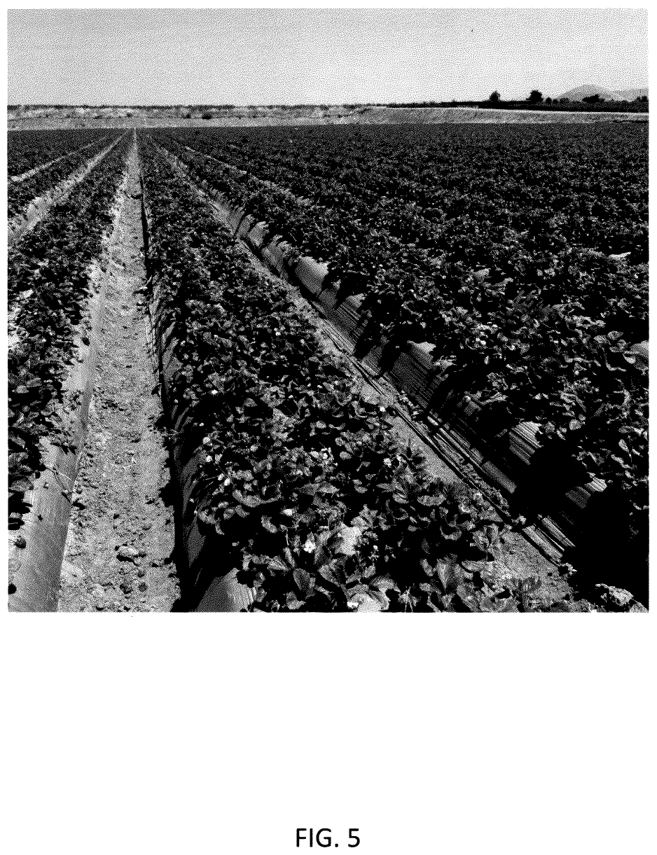

FIG. 5 shows a typical field of plants and fruit mid-season.

### DETAILED BOTANICAL DESCRIPTION OF THE PLANT

‘CBC008’ is a new and distinct day-neutral cultivar of strawberry, genus, and species Fragaria x ananassa. Plants of ‘CBC008’ bloom continuously in higher elevation plantings as 26 well as the heat of low elevation nursery locations such as French Camp, CA, consistent with other day-neutral varieties. The plants of the new cultivar have a moderate vigor.

‘CBC008’ exhibits several characteristics which are improvements over one or both of its parent varieties, and other cultivars known to the Inventor. These characteristics are compared with other cultivars in the following discussion and tables.

The fruit size of ‘CBC008’ is medium sized, slightly larger than its parents. The fruit size of ‘CBC008’ is still within an ideal range since it is not too large to be damaged in shipping containers, but not too small to allow for efficient picking and packing. Storability of the fruit has not been measured but in informal observations the fruit retains good physical characteristics and appearance for several days after harvest at room temperature and up to at least a week in common refrigerator conditions of roughly 3° C.

The data set for ‘CBC008’ set forth below was collected in May 2025 from plants grown at a test plot in Watsonville, CA. Yield, size, firmness data is either averaged over the entire 2024-2025 growing season or presented as monthly totals at the same location. Description data was collected on five-month-old plants.

The observed characteristics of ‘CBC008’ are set forth in Table 1, 2, 3, and 4 below. Color terminology, where noted, is in accordance with the Munsell Colorchart. The observed characteristics of ‘CBC008’ are compared to the closest cultivar known to the Inventor, in Table 2, 3, and 4. Table 5 sets forth the test results of DNA fingerprinting using eight SSR markers.

TABLE 1
Detailed Description of Characteristics of ‘CBC008’

| | |
|---|---|
| SPECIFICATION: | |
| Genus/Species | Fragaria X ananassa |
| Market Name | Strawberry |
| PARENTS: | |
| Female | ‘Sabrina’ |
| Male | ‘Driscoll Camarillo’ |
| PLANT: | |
| Type | Day-neutral |
| Growth Habit | Upright |
| Foliage density | Moderate |
| Vigor | Moderate |
| Height | Average 22.6 cm; range 19-26 cm |
| Width | Average 28.8 cm; range 25-33 cm |
| Disease tolerance | Test plots have shown tolerance to |
| | Macrophomina phaseolina, |
| | Fusarium oxysporum f. sp fragariae |
| | and Verticillium dahlia. |
| LEAF: | |
| Width | 15 cm |
| Color | Adaxial surface: 5 GY 3/4 |
| | Abaxial surface: 5 GY 5/4 |
| Pubescence | Slight |
| Glossiness | Medium |
| Variegation | Not present |
| TERMINAL LEAFLET | |
| Length | Average 58 mm; range 55-61 mm |
| Width | Average 47 mm; range 45-49 mm |
| Ratio (length/width) | 1.23 |
| Margins | Crenate to serrate |
| Leaflet shape | Ovate |
| Base shape | Cordate |
| Cross-section shape | Concave |
| PETIOLE | |
| Pubescence | Slight |
| Petiole color | 5 GY 7/4 |
| Petiole length | Average 15.5 cm; range 13-18 cm |
| Petiole diameter | Average 2.2 mm; range 1.8-2.6 mm |
| STOLON | |
| Anthocyanin coloration | Present, medium |
| Pubescence | Slight |
| Diameter | Average 2 mm; range 1.5-2.5 mm |
| STIPULE | |
| Anthocyanin coloration | Present, medium |
| Length | Average 10.5 mm; range 9-12 mm |
| Width | Average 6 mm; range 5-7 mm |
| INFLORESCENCE: | |
| Flowering | Day-neutral |
| Position | At or slightly above canopy |
| Petal arrangement | Overlapping |
| Calyx size | Large |
| Number of blooms | 12 |
| Length | 18 cm |
| Flower diameter | Average 22 mm; range 20-24 mm |
| Petal number | Average 5; range 5-6 |
| Petal length | Average 10 mm; range 9-11 mm |
| Petal width | Average 9 mm; range 8-10 mm |
| Ratio (length/width) | 1.11 |
| Petal color | 7.5Y 9/2 |
| Calyx color | Adaxial surface: 5 GY 4/6 |
| | Abaxial surface: 5 GY 5/4 |
| Stamens length | Average 20 mm; range 18-24 mm |
| PEDICEL | |
| Pubescence | Slight |
| Length | Average 93 mm; range 85-100 mm |
| Width | Average 2.2 mm; range 2-2.5 mm |
| Ratio (length/width) | 42.3 |
| Color | 5 GY 6/6 |
| FRUIT: | |
| Bearing | Day-neutral |
| Individual fruit weight | Seasonal average: 33.2 g |
| Shape | Broad conic to cordate |
| Achenes | Mostly level to slightly protuding |
| Glossiness | High gloss |
| External color evenness | uniform |
| External color | 7.5R 4/12 |
| Internal color | 7.5 R 5/8 |
| Band at top without achenes | Slight |
| Fruit Yield | Grams per plant: 3006 g |
| Firmness | 0.51 kg/force |
| Achene color | 10 YR 6/6 |

TABLE 2
Marketable yield of ‘CBC008’ compared to other cultivars known
to the Inventor in Watsonville, CA during the 2024-2025 season:
‘Cabrillo’, U.S. Plant Pat. No. 27,830. and
‘Monterey’, U.S. Plant Pat. No. 19,767.

| | Marketable yield (g/plant) | | | | | |
|---|---|---|---|---|---|---|
| Cultivar | Apr. | May | Jun. | Jul. | Aug. | Total |
| ‘CBC008’ | 448 | 586 | 770 | 973 | 228 | 3006 |
| ‘Cabrillo’ | 186 | 415 | 717 | 800 | 218 | 2336 |
| ‘Monterey’ | 195 | 485 | 648 | 600 | 178 | 2105 |

TABLE 3
Size of ‘CBC008’ compared to other cultivars known
to the Inventor in Watsonville, CA during the 2024-2025 season.

| | Fruit size (g/fruit) | | | | | |
|---|---|---|---|---|---|---|
| Cultivar | Apr. | May | Jun. | Jul. | Aug. | Average |
| ‘CBC008’ | 43.1 | 37.1 | 33.7 | 30.8 | 21.1 | 33.2 |
| ‘Cabrillo’ | 37.0 | 37.9 | 33.5 | 27.9 | 23.6 | 32.0 |
| ‘Monterey’ | 41.2 | 39.2 | 30.0 | 23.2 | 19.7 | 30.7 |

TABLE 4
Firmness of ‘CBC008’ compared to other cultivars known
to the inventor in Watsonville, CA during the 2024-2025 season.
Firmness (kg/force)

| Cultivar | May | Jun. | Jul. | Average |
|---|---|---|---|---|
| CBC008’ | 0.56 | 0.50 | 0.47 | 0.51 |
| ‘Cabrillo’ | 0.60 | 0.55 | 0.56 | 0.57 |
| ‘Monterey’ | 0.56 | 0.51 | 0.50 | 0.52 |

TABLE 5
DNA finger printing results of eight SSR markers.

| | Allelic Fingerprint Analysis | | | | | | | |
|---|---|---|---|---|---|---|---|---|
| Cultivar | M1 | M2 | M3 | M4 | M5 | M6 | M7 | M8 |
| ‘CBC008' | 224, | 259, | 188, | 279, | 156 | 242, | 121, | 213, |
| | 229 | 264, | 190, | 281, | | 251, | 124, | 215, |
| | | 269, | 216, | 289 | | 257, | 130, | 222, |
| | | 271, | 232 | | | 262, | 138, | 228 |
| | | 275 | | | | 264 | 146 | |

I claim:

1. A new and distinct strawberry plant named ‘CBC008’ as described and illustrated herein.
